(19) United States
(12) Patent Application Publication
(10) Pub. No.: US 2003/0044949 A1
(43) Pub. Date: Mar. 6, 2003

(54) Polypeptides having gamma-glutamyl transpeptidase activity and nucleic acids encoding same

(75) Inventors: Regine Behr, Roseville, CA (US); Alan Sloma, Davis, CA (US)

Correspondence Address: NOVOZYMES BIOTECH, INC., 1445 DREW AVE, DAVIS, CA, 95616, US

(73) Assignee: Novozymes Biotech, Inc., Davis, CA (US)

(21) Appl. No.: 10/107,649

(22) Filed: Mar. 27, 2002

Related U.S. Application Data
(60) Provisional application No. 60/279,374, filed on Mar. 27, 2001.

(51) Int. Cl. C12N 9/10; C12N 9/64; C07H 21/04; C12P 21/02; C12N 5/06

(52) U.S. Cl. 435/193; 435/69.1; 435/320.1; 435/325; 536/23.2; 435/226

(57) ABSTRACT

The present invention relates to isolated polypeptides having gamma-glutamyl transpeptidase activity and isolated nucleic acid sequences encoding the polypeptides. The invention also relates to nucleic acid constructs, vectors, and host cells comprising the nucleic acid sequences as well as methods for producing and using the polypeptides.

### CROSS-REFERENCE TO RELATED APPLICATION

This application claims priority from pending U.S. provisional application Serial No. 60/482,751, filed Mar. 27, 2001, which application is fully incorporated herein by reference.

### BACKGROUND OF THE INVENTION

1. Field of the Invention

The present invention relates to isolated polypeptides having gamma-glutamyl transpeptidase activity and isolated nucleic acid sequences encoding the polypeptides. The invention also relates to nucleic acid constructs, vectors, and host cells comprising the nucleic acid sequences as well as methods for producing and using the polypeptides.

2. Description of the Related Art

Gamma-glutamyl transpeptidase (E.C. 2.3.2.2) plays a major role in glutathione metabolism where the enzyme catalyzes the transfer of the gamma-glutamyl group from gamma-glutamyl compounds to amino acids, peptide acceptors, or water (Tate and Meister, 1981, Mol. Cell. Biochem. 39: 357-368). For example, gamma-glutamyl transpeptidase catalyzes (1) the hydrolysis of glutathione to produce glutamic acid or (2) the transfer of the gamma-glutamyl group of glutathione to an amino acid or peptide as follows:

(1) γ-Glu-Cys-Gly+H₂O Glu+Cys-Gly

(2) γ-Glu-Cys-Gly+Peptide -Glu-Peptide+Cys-Gly

Gamma-glutamyl transpeptidases have been reported from Bacillus licheniformis (JP 4281787), Bacillus natto (JP 2065777 A), Bacillus subtilis (U.S. Pat. Nos. 4,990,444; 5,153,120; JP94095936), and Bacillus subtilis-natto (Ogawa et al., 1991, Agricultural and Biological Chemistry 55: 2971-2977; Hara et al., 1985, Journal of the Faculty of Agriculture Kyushu University 30: 95-106).

Genes encoding gamma-glutamyl transpeptidases have been cloned from Bacillus anthracis (Uchida et al., 1993, Molecular Microbiology 9: 487-496); Bacillus subtilis (Xu and Strauch, 1996, Journal of Bacteriology 178: 4319-4322; Hara et al., 1992, Applied Microbiology and Biotechnology 37: 211-215); and Bacillus subtilis-natto (Hara et al., 1992, Journal of the Faculty of Agriculture Kyushu University 36: 209-218).

It is an object of the present invention to provide improved polypeptides having gamma-glutamyl transpeptidase activity and nucleic acids encoding the polypeptides.

### SUMMARY OF THE INVENTION

The present invention relates to isolated polypeptides having gamma-glutamyl transpeptidase activity selected from the group consisting of:

(a) a polypeptide having an amino acid sequence which has at least 65% identity with amino acids 31 to 604 of SEQ ID NO. 2;

(b) a polypeptide encoded by a nucleic acid sequence which hybridizes under low, stringency conditions with (i) nucleotides 91 to 1812 of SEQ ID NO. 1, (ii) a subsequence of (i) of at least 100 nucleotides, or (iii) a complementary strand of (i) or (ii);and

(c) a fragment of (a) or (b), that has gamma-glutamyl transpeptidase activity.

The present invention also relates to isolated nucleic acid sequences encoding the polypeptides and to nucleic acid constructs, vectors, and host cells comprising the nucleic acid sequences as well as methods for producing and using the polypeptides.

### BRIEF DESCRIPTION OF THE FIGURE

FIG. 1 shows the genomic DNA sequence and the deduced amino acid sequence of a Bacillus agaradhaerens NCIMB 40482 gamma-glutamyl transpeptidase (SEQ ID NOS. 1 and 2, respectively).

### DETAILED DESCRIPTION OF THE INVENTION

Polypeptides Having Gamma-Glutamyl Transpeptidase Activity

The term “gamma-glutamyl transpeptidase activity” is defined herein as a (5-L-glutamyl)-peptide:amino acid 5-glutamyltransferase activity which catalyzes the conversion of (5-L-glutamyl)-peptide and an amino acid to peptide and 5-L-glutamyl amino acid. For purposes of the present invention, gamma-glutamyl transpeptidase activity is determined according to the procedure described by Orlowski and Meister, 1963, Biochim. Biphys. Acta 73: 679-681, using gamma-glutamyl-p-nitroanilide as substrate. One unit of gamma-glutamyl transpeptidase activity is defined as 1.0 μmole of p-nitroaniline produced per minute at 25° C., pH 8.5.

In a first embodiment, the present invention relates to isolated polypeptides having an amino acid sequence which has a degree of identity to amino acids 31 to 604 of SEQ ID NO. 2 (i.e., the mature polypeptide) of at least about 65%, preferably at least about 70%, more preferably at least about 80%, even more preferably at least about 85%, even more preferably at least about 90%, most preferably at least about 95%, and even most preferably at least about 97%, which have gamma-glutamyl transpeptidase activity (hereinafter “homologous polypeptides”). In a preferred embodiment, the homologous polypeptides have an amino acid sequence which differs by five amino acids, preferably by four amino acids, more preferably by three amino acids, even more preferably by two amino acids, and most preferably by one amino acid from amino acids 31 to 604 of SEQ ID NO. 2. For purposes of the present invention, the degree of identity between two amino acid sequences is determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters were Ktuple=1, gap penalty-3, windows=5, and diagonals=5.

Preferably, the polypeptides of the present invention comprise the amino acid sequence of SEQ ID NO. 2 or an allelic variant thereof; or a fragment thereof that has gamma-glutamyl transpeptidase activity. In a more preferred embodiment, the polypeptide of the present invention comprises the amino acid sequence of SEQ ID NO. 2. In another preferred embodiment, the polypeptide of the present invention comprises amino acids 31 to 604 of SEQ ID NO. 2, or an allelic variant thereof; or a fragment thereof that has gamma-glutamyl transpeptidase activity. In another preferred embodiment, the polypeptide of the present invention comprises amino acids 31 to 604 of SEQ ID NO. 2. In another preferred embodiment, the polypeptide of the present invention consists of the amino acid sequence of SEQ ID NO. 2 or an allelic variant thereof; or a fragment thereof that has gamma-glutamyl transpeptidase activity. In another preferred embodiment, the polypeptide of the present invention consists of the amino acid sequence of SEQ ID NO. 2. In another preferred embodiment, the polypeptide consists of amino acids 31 to 604 of SEQ ID NO. 2 or an allelic variant thereof, or a fragment thereof that has gamma-glutamyl transpeptidase activity. In another preferred embodiment, the polypeptide consists of amino acids 31 to 604 of SEQ ID NO. 2.

A fragment of SEQ ID NO. 2 is a polypeptide having one or more amino acids deleted from the amino and/or carboxyl terminus of this amino acid sequence. Preferably, a fragment contains at least 500 amino acid residues, more preferably at least 525 amino acid residues, and most preferably at least 550 amino acid residues.

An allelic variant denotes any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

In a second embodiment, the present invention relates to isolated polypeptides having gamma-glutamyl transpeptidase activity which are encoded by nucleic acid sequences which hybridize under very low stringency conditions, preferably low stringency conditions, more preferably medium stringency conditions, more preferably medium-high stringency conditions, even more preferably high stringency conditions, and most preferably very high stringency conditions with a nucleic acid probe which hybridizes under the same conditions with (i) nucleotides 91 to 1812 of SEQ ID NO. 1, (ii) a subsequence of (i), or (iii) a complementary strand of (i), or (ii) (J. Sambrook, E. F. Fritsch, and T. Maniatus, 1989, Molecular Cloning, A Laboratory Manual, 2d edition, Cold Spring Harbor, N.Y.). The subsequence of SEQ ID NO. 1 may be at least 100 nucleotides or preferably at least 200 nucleotides. Moreover, the subsequence may encode a polypeptide fragment which has gamma-glutamyl transpeptidase activity. The polypeptides may also be allelic variants or fragments of the polypeptides that have gamma-glutamyl transpeptidase activity.

The nucleic acid sequence of SEQ ID NO. 1 or a subsequence thereof, as well as the amino acid sequence of SEQ ID NO. 2 or a fragment thereof, may be used to design a nucleic acid probe to identify and clone DNA encoding polypeptides having gamma-glutamyl transpeptidase activity from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 15, preferably at least 25, and more preferably at least 35 nucleotides in length. Longer probes can also be used. Both DNA and RNA probes can be used. The probes are typically labeled for detecting the corresponding gene (for example, with ³²P, ³H, ³⁵S, biotin, or avidin). Such probes are encompassed by the present invention.

Thus, a genomic DNA or cDNA library prepared from such other organisms may be screened for DNA which hybridizes with the probes described above and which encodes a polypeptide having gamma-glutamyl transpeptidase activity. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA which is homologous with SEQ ID NO. 1 or a subsequence thereof, the carrier material is used in a Southern blot. For purposes of the present invention, hybridization indicates that the nucleic acid sequence hybridizes to a labeled nucleic acid probe corresponding to the nucleic acid sequence shown in SEQ ID NO. 1, its complementary strand, or a subsequence thereof, under very low to very high stringency conditions. Molecules to which the nucleic acid probe hybridizes under these conditions are detected using X-ray film.

In a preferred embodiment, the nucleic acid probe is a nucleic acid sequence which encodes the polypeptide of SEQ ID NO. 2, or a subsequence thereof. In another preferred embodiment, the nucleic acid probe is SEQ ID NO. 1. In another preferred embodiment, the nucleic acid probe is the mature polypeptide coding region of SEQ ID NO. 1. In another preferred embodiment, the nucleic acid probe is the nucleic acid sequence contained in plasmid pRB155 which is contained in Escherichia coli NRRL B-30340, wherein the nucleic acid sequence encodes a polypeptide having gamma-glutamyl transpeptidase activity. In another preferred embodiment, the nucleic acid probe is the mature polypeptide coding region contained in plasmid pRB155 which is contained in Escherichia coli NRRL B-30340.

For long probes of at least 100 nucleotides in length, very low to very high stringency conditions are defined as prehybridization and hybridization at 42° C. in 5× SSPE, 0.3% SDS, 200 μg/ml sheared and denatured salmon sperm DNA, and either 25% formamide for very low and low stringencies, 35% formamide for medium and medium-high stringencies, or 50% formamide for high and very high stringencies, following standard Southern blotting procedures.

For long probes of at least 100 nucleotides in length, the carrier material is finally washed three times each for 15 minutes using 2× SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

For short probes which are about 15 nucleotides to about 70 nucleotides in length, stringency conditions are defined as prehybridization, hybridization, and washing post-hybridization at about 5° C. to about 10° C. below the calculated Tm using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1× Denhardt's solution, 1 mM sodium pyrophosphate, 1 mM sodium monobasic phosphate, 0.1 mM ATP, and 0.2 mg of yeast RNA per ml following standard Southern blotting procedures.

For short probes which are about 15 nucleotides to about 70 nucleotides in length, the carrier material is washed once in 6× SCC plus 0.1% SDS for 15 minutes and twice each for 15 minutes using 6× SSC at 5° C. to 10° C. below the calculated Tm.

In a third embodiment, the present invention relates to variants of the polypeptide having an amino acid sequence of SEQ ID NO. 2 comprising a substitution, deletion, and/or insertion of one or more amino acids.

The amino acid sequences of the variant polypeptides may differ from the amino acid sequence of SEQ ID NO. 2 or the mature polypeptide thereof by an insertion or deletion of one or more amino acid residues and/or the substitution of one or more amino acid residues by different amino acid residues. Preferably, amino acid changes are of a minor nature, that is conservative amino acid substitutions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter the specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly as well as these in reverse.

In a fourth embodiment, the present invention relates to isolated polypeptides having immunochemical identity or partial immunochemical identity to the polypeptide having the amino acid sequence of SEQ ID NO. 2 or the mature polypeptide thereof. The immunochemical properties are determined by immunological cross-reaction identity tests by the well-known Ouchterlony double immunodiffusion procedure. Specifically, an antiserum containing polyclonal antibodies which are immunoreactive or bind to epitopes of the polypeptide having the amino acid sequence of SEQ ID NO. 2 or the mature polypeptide thereof are prepared by immunizing rabbits (or other rodents) according to the procedure described by Harboe and Ingild, In N. H. Axelsen, J. Krøll, and B. Weeks, editors, A Manual of Quantitative Immunoelectrophoresis, Blackwell Scientific Publications, 1973, Chapter 23, or Johnstone and Thorpe, Immunochemistry in Practice, Blackwell Scientific Publications, 1982 (more specifically pages 27-31). A polypeptide having immunochemical identity is a polypeptide which reacts with the antiserum in an identical fashion such as total fusion of precipitates, identical precipitate morphology, and/or identical electrophoretic mobility using a specific immunochemical technique. A further explanation of immunochemical identity is described by Axelsen, Bock, and Krøll, In N. H. Axelsen, J. Krøll, and B. Weeks, editors, A Manual of Quantitative Immunoelectrophoresis, Blackwell Scientific Publications, 1973, Chapter 10. A polypeptide having partial immunochemical identity is a polypeptide which reacts with the antiserum in a partially identical fashion such as partial fusion of precipitates, partially identical precipitate morphology, and/or partially identical electrophoretic mobility using a specific immunochemical technique. A further explanation of partial immunochemical identity is described by Bock and Axelsen, In N. H. Axelsen, J. Krøll, and B. Weeks, editors, A Manual of Quantitative Immunoelectrophoresis, Blackwell Scientific Publications, 1973, Chapter 11.

The antibody may also be a monoclonal antibody. Monoclonal antibodies may be prepared and used, e.g., according to the methods of E. Harlow and D. Lane, editors, 1988, Antibodies, A Laboratory Manual, Cold Spring Harbor Press, Cold Spring Harbor, N.Y.

The polypeptides of the present invention have at least 20%, preferably at least 40%, more preferably at least 60%, even more preferably at least 80%, even more preferably at least 90%, and most preferably at least 100% of the gamma-glutamyl transpeptidase activity of the mature polypeptide of SEQ ID NO. 2.

A polypeptide of the present invention may be obtained from microorganisms of any genus. For purposes of the present invention, the term “obtained from” as used herein in connection with a given source shall mean that the polypeptide encoded by the nucleic acid sequence is produced by the source or by a cell in which the nucleic acid sequence from the source has been inserted. In a preferred embodiment, the polypeptide is secreted extracellularly.

A polypeptide of the present invention may be a bacterial polypeptide. For example, the polypeptide may be a gram positive bacterial polypeptide such as a Bacillus polypeptide, e.g., a Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis polypeptide; or a Streptomyces polypeptide, e.g., a Streptomyces lividans or Streptomyces murinus polypeptide; or a gram negative bacterial polypeptide, e.g., an E. coli or a Pseudomonas sp. polypeptide.

In a more preferred embodiment, the polypeptide is a Bacillus agaradhaerens polypeptide, and most preferably a Bacillus agaradhaerens NCIMB 40482 polypeptide, e.g., the polypeptide with the amino acid sequence of SEQ ID NO. 2.

Strains of these species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

Furthermore, such polypeptides may be identified and obtained from other sources including microorganisms isolated from nature (e.g., soil, composts, water, etc.) using the above-mentioned probes. Techniques for isolating microorganisms from natural habitats are well known in the art. The nucleic acid sequence may then be derived by similarly screening a genomic or cDNA library of another microorganism. Once a nucleic acid sequence encoding a polypeptide has been detected with the probe(s), the sequence may be isolated or cloned by utilizing techniques which are known to those of ordinary skill in the art (see, e.g., Sambrook et al., 1989, supra).

As defined herein, an “isolated” polypeptide is a polypeptide which is essentially free of other non-gamma-glutamyl transpeptidase polypeptides, e.g., at least about 20% pure, preferably at least about 40% pure, more preferably about 60% pure, even more preferably about 80% pure, most preferably about 90% pure, and even most preferably about 95% pure, as determined by SDS-PAGE.

Polypeptides encoded by nucleic acid sequences of the present invention also include fused polypeptides or cleavable fusion polypeptides in which another polypeptide is fused at the N-terminus or the C-terminus of the polypeptide or fragment thereof. A fused polypeptide is produced by fusing a nucleic acid sequence (or a portion thereof) encoding another polypeptide to a nucleic acid sequence (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator.

Nucleic Acid Sequences

The present invention also relates to isolated nucleic acid sequences which encode a polypeptide of the present invention. In a preferred embodiment, the nucleic acid sequence is set forth in SEQ ID NO. 1. In another more preferred embodiment, the nucleic acid sequence is the sequence contained in plasmid pRB155 that is contained in Escherichia coli NRRL B-30340. In another preferred embodiment, the nucleic acid sequence is the mature polypeptide coding region of SEQ ID NO. 1. In another more preferred embodiment, the nucleic acid sequence is the mature polypeptide coding region contained in plasmid pRB155 that is contained in Escherichia coli NRRL B-30340. The present invention also encompasses nucleic acid sequences which encode a polypeptide having the amino acid sequence of SEQ ID NO. 2 or the mature polypeptide thereof, which differ from SEQ ID NO. 1 by virtue of the degeneracy of the genetic code. The present invention also relates to subsequences of SEQ ID NO. 1 which encode fragments of SEQ ID NO. 2 that have gamma-glutamyl transpeptidase activity.

A subsequence of SEQ ID NO. 1 is a nucleic acid sequence encompassed by SEQ ID NO. 1 except that one or more nucleotides from the 5′ and/or 3′ end have been deleted. Preferably, a subsequence contains at least 1500 nucleotides, more preferably at least 1575 nucleotides, and most preferably at least 1650 nucleotides.

The present invention also relates to mutant nucleic acid sequences comprising at least one mutation in the mature polypeptide coding sequence of SEQ ID NO. 1, in which the mutant nucleic acid sequence encodes a polypeptide which consists of amino acids 31 to 604 of SEQ ID NO. 2.

The techniques used to isolate or clone a nucleic acid sequence encoding a polypeptide are known in the art and include isolation from genomic DNA, preparation from cDNA, or a combination thereof. The cloning of the nucleic acid sequences of the present invention from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleic acid sequence-based amplification (NASBA) may be used. The nucleic acid sequence may be cloned from a strain of Bacillus, or another or related organism and thus, for example, may be an allelic or species variant of the polypeptide encoding region of the nucleic acid sequence.

The term “isolated nucleic acid sequence” as used herein refers to a nucleic acid sequence which is essentially free of other nucleic acid sequences, e.g., at least about 20% pure, preferably at least about 40% pure, more preferably at least about 60% pure, even more preferably at least about 80% pure, and most preferably at least about 90% pure as determined by agarose electrophoresis. For example, an isolated nucleic acid sequence can be obtained by standard cloning procedures used in genetic engineering to relocate the nucleic acid sequence from its natural location to a different site where it will be reproduced. The cloning procedures may involve excision and isolation of a desired nucleic acid fragment comprising the nucleic acid sequence encoding the polypeptide, insertion of the fragment into a vector molecule, and incorporation of the recombinant vector into a host cell where multiple copies or clones of the nucleic acid sequence will be replicated. The nucleic acid sequence may be of genomic, cDNA, RNA, semisynthetic, synthetic origin, or any combinations thereof.

The present invention also relates to nucleic acid sequences which have a degree of homology to the mature polypeptide coding sequence of SEQ ID NO. 1 (i.e., nucleotides 91 to 1812) of at least about 65%, preferably about 70%, preferably about 80%, preferably about 85%, more preferably about 90%, even more preferably about 95%, and most preferably about 97% homology, which encode an active polypeptide. For purposes of the present invention, the degree of homology between two nucleic acid sequences is determined by the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters were Ktuple=3, gap penalty-3, and windows=20.

Modification of a nucleic acid sequence encoding a polypeptide of the present invention may be necessary for the synthesis of polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variant sequence may be constructed on the basis of the nucleic acid sequence presented as the polypeptide encoding part of SEQ ID NO. 1, e.g., a subsequence thereof, and/or by introduction of nucleotide substitutions which do not give rise to another amino acid sequence of the polypeptide encoded by the nucleic acid sequence, but which correspond to the codon usage of the host organism intended for production of the enzyme, or by introduction of nucleotide substitutions which may give rise to a different amino acid sequence. For a general description of nucleotide substitution, see, e.g., Ford et al., 1991, Protein Expression and Purification 2: 95-107.

It will be apparent to those skilled in the art that such substitutions can be made outside the regions critical to the function of the molecule and still result in an active polypeptide. Amino acid residues essential to the activity of the polypeptide encoded by the isolated nucleic acid sequence of the invention, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for gamma-glutamyl transpeptidase activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

The present invention also relates to isolated nucleic acid sequences encoding a polypeptide of the present invention, which hybridize under very low stringency conditions, preferably low stringency conditions, more preferably medium stringency conditions, more preferably medium-high stringency conditions, even more preferably high stringency conditions, and most preferably very high stringency conditions with a nucleic acid probe which hybridizes under the same conditions with the nucleic acid sequence of SEQ ID NO. 1 or its complementary strand; or allelic variants and subsequences thereof (Sambrook et al., 1989, supra), as defined herein.

The present invention also relates to isolated nucleic acid sequences produced by (a) hybridizing a DNA under very low, low, medium, medium-high, high, or very high stringency conditions with (i) nucleotides 91 to 1812 of SEQ ID NO. 1, (ii) a subsequence of (i), or (iii) a complementary strand of (i) or (ii); and (b) isolating the nucleic acid sequence. The subsequence is preferably a sequence of at least 100 nucleotides such as a sequence which encodes a polypeptide fragment which has gamma-glutamyl transpeptidase activity.

Methods for Producing Mutant Nucleic Acid Sequences

The present invention further relates to methods for producing a mutant nucleic acid sequence, comprising introducing at least one mutation into the mature polypeptide coding sequence of SEQ ID NO. 1 or a subsequence thereof, wherein the mutant nucleic acid sequence encodes a polypeptide which consists of amino acids 31 to 604 of SEQ ID NO. 2 or a fragment thereof which has gamma-glutamyl transpeptidase activity.

The introduction of a mutation into the nucleic acid sequence to exchange one nucleotide for another nucleotide may be accomplished by site-directed mutagenesis using any of the methods known in the art. Particularly useful is the procedure which utilizes a supercoiled, double stranded DNA vector with an insert of interest and two synthetic primers containing the desired mutation. The oligonucleotide primers, each complementary to opposite strands of the vector, extend during temperature cycling by means of Pfu DNA polymerase. On incorporation of the primers, a mutated plasmid containing staggered nicks is generated. Following temperature cycling, the product is treated with DpnI which is specific for methylated and hemimethylated DNA to digest the parental DNA template and to select for mutation-containing synthesized DNA. Other procedures known in the art may also be used.

Nucleic Acid Constructs

The present invention also relates to nucleic acid constructs comprising a nucleic acid sequence of the present invention operably linked to one or more control sequences which direct the expression of the coding sequence in a suitable host cell under conditions compatible with the control sequences. Expression will be understood to include any step involved in the production of the polypeptide including, but not limited to, transcription, post-transcriptional modification, translation, post-translational modification, and secretion.

“Nucleic acid construct” is defined herein as a nucleic acid molecule, either single- or double-stranded, which is isolated from a naturally occurring gene or which has been modified to contain segments of nucleic acid combined and juxtaposed in a manner that would not otherwise exist in nature. The term nucleic acid construct is synonymous with the term expression cassette when the nucleic acid construct contains all the control sequences required for expression of a coding sequence of the present invention. The term “coding sequence” is defined herein as a nucleic acid sequence which directly specifies the amino acid sequence of its protein product. The boundaries of a genomic coding sequence are generally determined by a ribosome binding site (prokaryotes) or by the ATG start codon (eukaryotes) located just upstream of the open reading frame at the 5′ end of the mRNA and a transcription terminator sequence located just downstream of the open reading frame at the 3′ end of the mRNA. A coding sequence can include, but is not limited to, DNA, cDNA, and recombinant nucleic acid sequences.

An isolated nucleic acid sequence encoding a polypeptide of the present invention may be manipulated in a variety of ways to provide for expression of the polypeptide. Manipulation of the nucleic acid sequence prior to its insertion into a vector may be desirable or necessary depending on the expression vector. The techniques for modifying nucleic acid sequences utilizing recombinant DNA methods are well known in the art.

The term “control sequences” is defined herein to include all components which are necessary or advantageous for the expression of a polypeptide of the present invention. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide. The term “operably linked” is defined herein as a configuration in which a control sequence is appropriately placed at a position relative to the coding sequence of the DNA sequence such that the control sequence directs the expression of a polypeptide.

The control sequence may be an appropriate promoter sequence, a nucleic acid sequence which is recognized by a host cell for expression of the nucleic acid sequence. The promoter sequence contains transcriptional control sequences which mediate the expression of the polypeptide. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

The control sequence may also be a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice may be used in the present invention.

The control sequence may also be a suitable leader sequence, a nontranslated region of an mRNA which is important for translation by the host cell. The leader sequence is operably linked to the 5′ terminus of the nucleic acid sequence encoding the polypeptide. Any leader sequence which is functional in the host cell of choice may be used in the present invention.

The control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used in the present invention.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, 1993, Microbiological Reviews 57: 109-137.

In a preferred embodiment, the signal peptide coding region is nucleotides 1 to 90 of SEQ ID NO. 1 which encode amino acids 1 to 30 of SEQ ID NO. 2.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE) and Bacillus subtilis neutral protease (nprT).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems.

Expression Vectors

The present invention also relates to recombinant expression vectors comprising a nucleic acid sequence of the present invention, a promoter, and transcriptional and translational stop signals. The various nucleic acid and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence of the present invention may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about the expression of the nucleic acid sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers which confer antibiotic resistance such as ampicillin, kanamycin, chloramphenicol or tetracycline resistance.

The vectors of the present invention preferably contain an element(s) that permits integration of the vector into the host cell's genome or autonomous replication of the vector in the cell independent of the genome.

For integration into the host cell genome, the vector may rely on the nucleic acid sequence encoding the polypeptide or any other element of the vector for integration of the vector into the genome by homologous or nonhomologous recombination. Alternatively, the vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus. The origin of replication may be one having a mutation which makes its functioning temperature-sensitive in the host cell (see, e.g., Ehrlich, 1978, Proceedings of the National Academy of Sciences USA 75: 1433).

More than one copy of a nucleic acid sequence of the present invention may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

Host Cells

The present invention also relates to recombinant host cells, comprising a nucleic acid sequence of the invention, which are advantageously used in the recombinant production of the polypeptides. A vector comprising a nucleic acid sequence of the present invention is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any unicellular microorganism, e.g., a prokaryote. Useful unicellular cells are bacterial cells such as gram positive bacteria including, but not limited to, a Bacillus cell, e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp. In a preferred embodiment, the bacterial host cell is a Bacillus lentus, Bacillus licheniformis, Bacillus stearothermophilus or Bacillus subtilis cell. In another preferred embodiment, the Bacillus cell is an alkalophilic Bacillus.

The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

Methods of Production

The present invention also relates to methods for producing a polypeptide of the present invention comprising (a) cultivating a strain, which in its wild-type form is capable of producing the polypeptide, to produce the polypeptide; and (b) recovering the polypeptide. Preferably, the strain is of the genus Bacillus, and more preferably Bacillus subtilis.

The present invention also relates to methods for producing a polypeptide of the present invention comprising (a) cultivating a host cell under conditions conducive for production of the polypeptide; and (b) recovering the polypeptide.

The present invention also relates to methods for producing a polypeptide of the present invention comprising (a) cultivating a host cell under conditions conducive for production of the polypeptide, wherein the host cell comprises a mutant nucleic acid sequence having at least one mutation in the mature polypeptide coding region of SEQ ID NO. 1, wherein the mutant nucleic acid sequence encodes a polypeptide which consists of amino acids 32 to 455 of SEQ ID NO. 2, and (b) recovering the polypeptide.

In the production methods of the present invention, the cells are cultivated in a nutrient medium suitable for production of the polypeptide using methods known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide may be recovered by methods known in the art. For example, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J. -C. Janson and Lars Ryden, editors, VCH Publishers, New York, 1989).

Plants

The present invention also relates to a transgenic plant, plant part, or plant cell which has been transformed with a nucleic acid sequence encoding a polypeptide having gamma-glutamyl transpeptidase activity of the present invention so as to express and produce the polypeptide in recoverable quantities. The polypeptide may be recovered from the plant or plant part. Alternatively, the plant or plant part containing the recombinant polypeptide may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as festuca, lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers. Also specific plant tissues, such as chloroplast, apoplast, mitochondria, vacuole, peroxisomes, and cytoplasm are considered to be a plant part. Furthermore, any plant cell, whatever the tissue origin, is considered to be a plant part.

Also included within the scope of the present invention are the progeny of such plants, plant parts and plant cells.

The transgenic plant or plant cell expressing a polypeptide of the present invention may be constructed in accordance with methods known in the art. Briefly, the plant or plant cell is constructed by incorporating one or more expression constructs encoding a polypeptide of the present invention into the plant host genome and propagating the resulting modified plant or plant cell into a transgenic plant or plant cell.

Conveniently, the expression construct is a nucleic acid construct which comprises a nucleic acid sequence encoding a polypeptide of the present invention operably linked with appropriate regulatory sequences required for expression of the nucleic acid sequence in the plant or plant part of choice. Furthermore, the expression construct may comprise a selectable marker useful for identifying host cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences is determined, for example, on the basis of when, where, and how the polypeptide is desired to be expressed. For instance, the expression of the gene encoding a polypeptide of the present invention may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV promoter may be used (Franck et al., 1980, Cell 21: 285-294). Organ-specific promoters may be, for example, a promoter from storage sink tissues such as seeds, potato tubers, and fruits (Edwards & Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant and Cell Physiology 39: 885-889), a Vicia faba promoter from the legumin B4 and the unknown seed protein gene from Vicia faba (Conrad et al., 1998, Journal of Plant Physiology 152: 708-711), a promoter from a seed oil body protein (Chen et al., 1998, Plant and Cell Physiology 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiology 102: 991-1000, the chlorella virus adenine methyltransferase gene promoter (Mitra and Higgins, 1994, Plant Molecular Biology 26: 85-93), or the aldP gene promoter from rice (Kagaya et al., 1995, Molecular and General Genetics 248: 668-674), or a wound inducible promoter such as the potato pin2 promoter (Xu et al., 1993, Plant Molecular Biology 22: 573-588).

A promoter enhancer element may also be used to achieve higher expression of the enzyme in the plant. For instance, the promoter enhancer element may be an intron which is placed between the promoter and the nucleotide sequence encoding a polypeptide of the present invention. For instance, Xu et al., 1993, supra disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Presently, Agrobacterium tumefaciens-mediated gene transfer is the method of choice for generating transgenic dicots (for a review, see Hooykas and Schilperoort, 1992, Plant Molecular Biology 19: 15-38). However it can also be used for transforming monocots, although other transformation methods are generally preferred for these plants. Presently, the method of choice for generating transgenic monocots is particle bombardment (microscopic gold or tungsten particles coated with the transforming DNA) of embryonic calli or developing embryos (Christou, 1992, Plant Journal 2: 275-281; Shimamoto, 1994, Current Opinion Biotechnology 5: 158-162; Vasil et al., 1992, Bio/Technology 10: 667-674). An alternative method for transformation of monocots is based on protoplast transformation as described by Omirulleh et al., 1993, Plant Molecular Biology 21: 415-428.

Following transformation, the transformants having incorporated therein the expression construct are selected and regenerated into whole plants according to methods well-known in the art.

The present invention also relates to methods for producing a polypeptide of the present invention comprising (a) cultivating a transgenic plant or a plant cell comprising a nucleic acid sequence encoding a polypeptide having gamma-glutamyl transpeptidase activity of the present invention under conditions conducive for production of the polypeptide; and (b) recovering the polypeptide.

Removal or Reduction of Gamma-Glutamyl Transpeptidase Activity

The present invention also relates to methods for producing a mutant cell of a parent cell, which comprises disrupting or deleting a nucleic acid sequence encoding the polypeptide or a control sequence thereof, which results in the mutant cell producing less of the polypeptide than the parent cell when cultivated under the same conditions.

The construction of strains which have reduced gamma-glutamyl transpeptidase activity may be conveniently accomplished by modification or inactivation of a nucleic acid sequence necessary for expression of the polypeptide having gamma-glutamyl transpeptidase activity in the cell. The nucleic acid sequence to be modified or inactivated may be, for example, a nucleic acid sequence encoding the polypeptide or a part thereof essential for exhibiting gamma-glutamyl transpeptidase activity, or the nucleic acid sequence may have a regulatory function required for the expression of the polypeptide from the coding sequence of the nucleic acid sequence. An example of such a regulatory or control sequence may be a promoter sequence or a functional part thereof, i.e., a part which is sufficient for affecting expression of the polypeptide. Other control sequences for possible modification are described above.

Modification or inactivation of the nucleic acid sequence may be performed by subjecting the cell to mutagenesis and selecting or screening for cells in which the gamma-glutamyl transpeptidase producing capability has been reduced. The mutagenesis, which may be specific or random, may be performed, for example, by use of a suitable physical or chemical mutagenizing agent, by use of a suitable oligonucleotide, or by subjecting the DNA sequence to PCR generated mutagenesis. Furthermore, the mutagenesis may be performed by use of any combination of these mutagenizing agents.

Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues.

When such agents are used, the mutagenesis is typically performed by incubating the cell to be mutagenized in the presence of the mutagenizing agent of choice under suitable conditions, and selecting for cells exhibiting reduced gamma-glutamyl transpeptidase activity or production.

Modification or inactivation of production of a polypeptide of the present invention may be accomplished by introduction, substitution, or removal of one or more nucleotides in the nucleic acid sequence encoding the polypeptide or a regulatory element required for the transcription or translation thereof. For example, nucleotides may be inserted or removed so as to result in the introduction of a stop codon, the removal of the start codon, or a change of the open reading frame. Such modification or inactivation may be accomplished by site-directed mutagenesis or PCR generated mutagenesis in accordance with methods known in the art. Although, in principle, the modification may be performed in vivo, i.e., directly on the cell expressing the nucleic acid sequence to be modified, it is preferred that the modification be performed in vitro as exemplified below.

An example of a convenient way to eliminate or reduce production by a host cell of choice is by gene replacement or gene interruption. In the gene interruption method, a nucleic acid sequence corresponding to the endogenous gene or gene fragment of interest is mutagenized in vitro to produce a defective nucleic acid sequence which is then transformed into the host cell to produce a defective gene. By homologous recombination, the defective nucleic acid sequence replaces the endogenous gene or gene fragment. It may be desirable that the defective gene or gene fragment also encodes a marker which may be used for selection of transformants in which the gene encoding the polypeptide has been modified or destroyed.

Alternatively, modification or inactivation of the nucleic acid sequence may be performed by established anti-sense techniques using a nucleotide sequence complementary to the polypeptide encoding sequence. More specifically, production of the polypeptide by a cell may be reduced or eliminated by introducing a nucleotide sequence complementary to the nucleic acid sequence encoding the polypeptide which may be transcribed in the cell and is capable of hybridizing to the polypeptide mRNA produced in the cell. Under conditions allowing the complementary anti-sense nucleotide sequence to hybridize to the polypeptide mRNA, the amount of polypeptide translated is thus reduced or eliminated.

It is preferred that the cell to be modified in accordance with the methods of the present invention is of microbial origin, for example, a fungal strain which is suitable for the production of desired protein products, either homologous or heterologous to the cell.

The present invention further relates to a mutant cell of a parent cell which comprises a disruption or deletion of a nucleic acid sequence encoding the polypeptide or a control sequence thereof, which results in the mutant cell producing less of the polypeptide than the parent cell.

The polypeptide-deficient mutant cells so created are particularly useful as host cells for the expression of homologous and/or heterologous polypeptides. Therefore, the present invention further relates to methods for producing a homologous or heterologous polypeptide comprising (a) cultivating the mutant cell under conditions conducive for production of the polypeptide; and (b) recovering the polypeptide. The term “heterologous polypeptides” is defined herein as polypeptides which are not native to the host cell, a native protein in which modifications have been made to alter the native sequence, or a native protein whose expression is quantitatively altered as a result of a manipulation of the host cell by recombinant DNA techniques.

In a further aspect, the present invention relates to a method for producing a protein product essentially free of gamma-glutamyl transpeptidase activity by fermentation of a cell which produces both a polypeptide of the present invention as well as the protein product of interest by adding an effective amount of an agent capable of inhibiting gamma-glutamyl transpeptidase activity to the fermentation broth before, during, or after the fermentation has been completed, recovering the product of interest from the fermentation broth, and optionally subjecting the recovered product to further purification.

In a further aspect, the present invention relates to a method for producing a protein product essentially free of gamma-glutamyl transpeptidase activity by cultivating the cell under conditions permitting the expression of the product, subjecting the resultant culture broth to a combined pH and temperature treatment so as to reduce the gamma-glutamyl transpeptidase activity substantially, and recovering the product from the culture broth. Alternatively, the combined pH and temperature treatment may be performed on an enzyme preparation recovered from the culture broth. The combined pH and temperature treatment may optionally be used in combination with a treatment with a gamma-glutamyl transpeptidase inhibitor.

In accordance with this aspect of the invention, it is possible to remove at least 60%, preferably at least 75%, more preferably at least 85%, still more preferably at least 95%, and most preferably at least 99% of the gamma-glutamyl transpeptidase activity. Complete removal of gamma-glutamyl transpeptidase activity may be obtained by use of this method.

The combined pH and temperature treatment is preferably carried out at a pH in the range of 6.5-7 and a temperature in the range of 25-40° C. for a sufficient period of time to attain the desired effect, where typically, 30 to 60 minutes is sufficient.

The methods used for cultivation and purification of the product of interest may be performed by methods known in the art.

The methods of the present invention for producing an essentially gamma-glutamyl transpeptidase-free product is of particular interest in the production of eukaryotic polypeptides, in particular fungal proteins such as enzymes. The enzyme may be selected from, e.g., an amylolytic enzyme, lipolytic enzyme, proteolytic enzyme, cellulytic enzyme, oxidoreductase, or plant cell-wall degrading enzyme. Examples of such enzymes include an aminopeptidase, amylase, amyloglucosidase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannosidase, oxidase, pectinolytic enzyme, peroxidase, phytase, phenoloxidase, polyphenoloxidase, proteolytic enzyme, ribonuclease, transferase, transglutaminase, or xylanase. The gamma-glutamyl transpeptidase-deficient cells may also be used to express heterologous proteins of pharmaceutical interest such as hormones, growth factors, receptors, and the like.

It will be understood that the term “eukaryotic polypeptides” includes not only native polypeptides, but also those polypeptides, e.g., enzymes, which have been modified by amino acid substitutions, deletions or additions, or other such modifications to enhance activity, thermostability, pH tolerance and the like.

In a further aspect, the present invention relates to a protein product essentially free from gamma-glutamyl transpeptidase activity which is produced by a method of the present invention.

Compositions

In a still further aspect, the present invention relates to compositions comprising a polypeptide of the present invention. Preferably, the compositions are enriched in a polypeptide of the present invention. In the present context, the term “enriched” indicates that the gamma-glutamyl transpeptidase activity of the composition has been increased, e.g., with an enrichment factor of 1.1.

The composition may comprise a polypeptide of the invention as the major enzymatic component, e.g., a mono-component composition. Alternatively, the composition may comprise multiple enzymatic activities, such as an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, or xylanase. The additional enzyme(s) may be producible by means of a microorganism belonging to the genus Aspergillus, preferably Aspergillus aculeatus, Aspergillus awamori, Aspergillus niger, or Aspergillus oryzae, or Trichoderma, Humicola, preferably Humicola insolens, or Fusariun, preferably Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sulphureum, Fusarium toruloseum, Fusarium trichothecioides, or Fusarium venenatum.

The polypeptide compositions may be prepared in accordance with methods known in the art and may be in the form of a liquid or a dry composition. For instance, the polypeptide composition may be in the form of a granulate or a microgranulate. The polypeptide to be included in the composition may be stabilized in accordance with methods known in the art.

Examples are given below of preferred uses of the polypeptide compositions of the invention. The dosage of the polypeptide composition of the invention and other conditions under which the composition is used may be determined on the basis of methods known in the art.

Uses

The present invention is also directed to methods for using the polypeptides having gamma-glutamyl transpeptidase activity.

The polypeptides may be used as a dough conditioner for increasing specific volume, for improving content, and for suppression of retrogradation of bread (see, Japanese Patent Application LOP Publn. No. 1985-2,135).

The polypeptides may also be used for the production of 7-amino-cephalosporamic acid from cephalosporin C (see, U.S. Pat. No. 4,990,444).

The polypeptides may also be used as a flavor enhancer by treatment of a protein-containing substance to release glutamic acid (see, for example, Tseng et al., 2000, Meat Science 55: 427-431; Tomita et al., 1988, Agricultural and Biological Chemistry 52: 1159-1163; Zhu et al., 1995, Applied Microbiology and Biotechnology 44: 277-282; and JP 7099923 A).

Signal Peptide

The present invention also relates to nucleic acid constructs comprising a gene encoding a protein operably linked to a nucleic acid sequence consisting of nucleotides 1 to 90 of SEQ ID NO. 1 encoding a signal peptide consisting of amino acids 1 to 30 of SEQ ID NO. 2, wherein the gene is foreign to the nucleic acid sequence.

The present invention also relates to recombinant expression vectors and recombinant host cells comprising such nucleic acid constructs.

The present invention also relates to methods for producing a protein comprising (a) cultivating such a recombinant host cell under conditions suitable for production of the protein; and (b) recovering the protein.

The first and second nucleic acid sequences may be operably linked to foreign genes individually with other control sequences or in combination with other control sequences. Such other control sequences are described supra. As noted earlier, where both signal peptide and propeptide regions are present at the amino terminus of a protein, the propeptide region is positioned next to the amino terminus of a protein and the signal peptide region is positioned next to the amino terminus of the propeptide region.

The protein may be native or heterologous to a host cell. The term “protein” is not meant herein to refer to a specific length of the encoded product and, therefore, encompasses peptides, oligopeptides, and proteins. The term “protein” also encompasses two or more polypeptides combined to form the encoded product. The proteins also include hybrid polypeptides which comprise a combination of partial or complete polypeptide sequences obtained from at least two different proteins wherein one or more may be heterologous or native to the host cell. Proteins further include naturally occurring allelic and engineered variations of the above mentioned proteins and hybrid proteins.

Preferably, the protein is a hormone or variant thereof, enzyme, receptor or portion thereof, antibody or portion thereof, or reporter. In a more preferred embodiment, the protein is an oxidoreductase, transferase, hydrolase, lyase, isomerase, or ligase. In an even more preferred embodiment, the protein is an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase or xylanase.

The gene may be obtained from any prokaryotic, eukaryotic, or other source.

The present invention is further described by the following examples which should not be construed as limiting the scope of the invention.

### EXAMPLES

Chemicals used as buffers and substrates were commercial products of at least reagent grade.

### Example 1

### Construction of Bacillus agaradhaerens Library

Bacillus agaradhaerens AC13 (NCIMB 40482) was used as source of chromosomal DNA for constructing a library. Strain E. coli JJC 128F′ araD139 A(ara-leu)7696 galE15 galK16 Δ(lac)X74 hsdr⁻ hsdm⁺ Str^{R} F′[lacl^{q} Δ(lacZ)M15 traD36] was used as a host to construct the genomic bank (Sorokin et al., 1996, Microbiology 142: 2005-2016).

Chromosomal DNA from Bacillus agaradhaerens AC13 was prepared as follows. Bacillus agaradhaerens was cultivated overnight at 37° C. in 125 ml shake flasks containing 25 ml of LB medium (Sambrook et al., Molecular Cloning: A Laboratory Manual, 2^{nd} ed., Cold Spring Harbor Laboratory Press, NY, 1989). The cells were harvested and treated with 10 μg of lysozyme per ml of 50 mM Tris-HCl pH 8.0, 50 mM EDTA, 25% sucrose. SDS was then added to a final concentration of 0.5% followed by proteinase K to 100 μg/ml. The mixture was incubated at 50° C. for 4 hours, and then extracted three times with water-saturated phenol-chloroform (1:1 v/v) at pH 8.0. After precipitation with two volumes of ethanol in 0.3 M sodium acetate pH 4.8, the DNA was removed with a glass rod, washed in 70% ethanol, and stored at −20° C. in water at 100 μg/ml.

Plasmid pSGMU2 (Errington, 1986, Journal of General Microbiology 132: 2953-2961) was used as a vector for constructing the chromosomal bank. pSGMU2 was isolated as follows. Cells of E. coli JJC 128F′, containing pSGMU2, were grown in 4 ml of 2× YT medium (Sambrook et al., 1989, supra) overnight. The cell pellet was resuspended in 100 μl of 50 mM glucose, 25 mM Tris/HCl pH 8.0, 10 mM EDTA solution (TE). Then a 100 μl volume of 10 mg/ml lysozyme was added. After 30 minutes 400 μl of 1% (w/v) SDS, 0.2 M NaOH were added. After cell lysis, 300 μl of 3 M sodium acetate pH 4.8, was added. After 30 minutes on ice, tubes were centrifuged at 13,000 rpm (5000× g) for 1 hour and 0.6 ml of isopropanol was added to the supernatant. After centrifugation as before for 10 minutes, the pellet was dissolved in 100 μl of water and then 100 μl of 9 M lithium chloride was added. After 1 hour at −20° C., tubes were centrifuged at 13,000 rpm (5000× g) for 10 minutes. The pellet was discarded and 500 μl of absolute ethanol was added to the supernatant. The pellet was redissolved in 300 μl of 0.3 M sodium acetate pH 4.8 and precipitated again. After dissolving the pellet in 100 μl of TE, the plasmid preparation was sufficiently pure for fluorescent sequencing.

A library with insert sizes in the range from 1 to 2 kb, was constructed by using pSGMU2. A 20 μg quantity of Bacillus agaradhaerens chromosomal DNA was sonicated using a VibraCell 72408 sonicator (Bioblock Scientific) at minimal amplitude for 10 seconds. The sonication was performed in 300 μl of Bal31 buffer (600 mM NaCl, 20 mM Tris-HCl pH 8.0, 12 mM CaCl₂, 12 mM MgCl₂, 1 mM EDTA) in a 1.5 ml Eppendorf tube. After sonication the chromosomal DNA was treated with Bal31 exonuclease (New England Biolabs, Inc., Beverly, Mass.) for 5 minutes at 25° C. After water-saturated phenol extraction and ethanol precipitation the DNA was treated by Klenow fragment of DNA polymerase I under the following conditions: 10 mM Tris HCl pH 7.6, 10 mM MgCl₂, 0.2 mM each dNTP, at 37° C. for 1 hour. After water-saturated phenol extraction and ethanol precipitation, the DNA was ligated with SmaI-digested pSGMU2 and treated with bacterial alkaline phosphatase. The ligation was performed in 10 mM Tris HCl pH 7.6, 10 mM MgCl_{2,} 1 mM DTT, 1 mM ATP at 10° C. for 6 hours. DNA from the ligation mixture was precipitated with ethanol in the presence of 1 mM glycogen at −20° C.

The DNA was then electroporated into E. coli JJC128F′ cells using 2.5 kV and 25 mF. The cells were plated on LB agar medium containing 50 μg/ml of ampicillin for selection of transformants and 20 μg/ml of 5-bromo-4-chloro-3-indolyl beta-D-galactopyranoside (XGAL) and 20 μg/ml of isopropyl beta-D-thiogalactopyranoside (IPTG) for selection of inserts. A total of-5000 plasmids were extracted from the white colonies and were sequenced by forward (M13-21) primer using a Perkin-Elmer Applied Biosystems Model 377 XL Automatic DNA Sequencer, Perkin-Elmer Applied Biosystems, Inc., Foster City, Calif.) with successful sequencing rate of about 90%.

Oligonucleotides were synthesized using a DNA Synthesizer “Oligo 1000” (Beckman-Coulter, Fullerton, Calif.). Primers used for Long Accurate PCR were 20-22-mers, chosen to contain 12 GC-bases.

Plasmid DNA for sequencing was prepared as described above. PCR products used for sequencing with dye terminators were purified by the Wizard™ PCR Preps kit (Promega, Madison, Wis.) or agarose gel electrophoresis. Forward and reverse PCR sequencing was performed using BigDye terminator Cycle Sequencing Ready Reaction Kit (Perkin-Elmer Applied Biosystems, Inc., Foster City, Calif.) and a “Perkin Elmer” 9600 thermal cycler or the “Catalyst” station (Perkin-Elmer Applied Biosystems, Inc., Foster City, Calif.). The fragment separation was conducted using an Applied Biosystems Model 377 XL Automatic DNA Sequencer.

A “Bacillus agaradhaerens genome tagging database” containing sequences obtained as described above was constructed. Vector sequences and contaminating E. coli sequences were removed with the crossmatch program from the Phred/Phrap package (Ewing and Green, 1998, Genome Research 8: 186-194). The sequences were assembled with Phrap also from the Phred/Phrap package. The assembled sequences were made available for homology searches using the BLAST version 1.4 programs (Altschul et al., 1990, J. Mol. Biol. 215: 403-410).

### Example 2

### Identifying a Bacillus agaradhaerens Gamma-Glutamyl Transpeptidase Homologue

The amino acid sequence of gamma-glutamyl transpeptidase (ggt) from Bacillus subtilis (http://bioweb.pasteur.fr/GenoList/SubtiList/genome.cgi) was used to search the Bacillus agaradhaerens genome tagging database. Using TBLASTN (TBLASTN 2.0.1 [Aug. 20, 1997], Altschul et al., 1997, Nucleic Acids Res. 25: 3389-3402), a hit was found. The homology showed a 34.96% identity in a 246 amino acids overlap.

### Example 3

### Preparation of Genomic DNA from Bacillus agaradhaerens AC13

Bacillus agaradhaerens AC13 (NCIMB 40482) was grown overnight at 37° C. in 13 ml of Luria-Bertani (LB) broth at pH 9.7. Genomic DNA was isolated using the QIAGEN bacterial genomic DNA isolation protocol (QIAGEN, Valencia, Calif.) according to the manufacturer's instructions.

### Example 4

### Preparation of a Probe of the Bacillus agaradhaerens Gamma-Glutamyl Transpeptidase Gene

Synthetic oligonucleotides RBggt1 and RBggt7R shown below were designed to PCR amplify a 320 bp probe (RBggt-1/7R) encompassing the region of homology described in Example 2. This probe was made using the Genius System PCR DIG Probe Synthesis (Roche Diagnostics Corporation, Indianapolis, Ind.) according to the manufacturer's instructions.

Primer RBggt1: 5′-GCCCATCCGTTAGCTGCAGA-3′ (SEQ ID NO. 3)

Primer RBggt7R: 5′-TTCAATGGCAGGGGCGATAAC-3′ (SEQ ID NO. 4)

The amplification reaction (50 μl) contained approximately 50 ng of Bacillus agaradhaerens AC13 genomic DNA, 0.5 μM of each primer, 200 μM each of dATP, dCTP, dGTP, and dTTP, 1× PCR buffer, 3 mM MgCl₂, and 0.625 units of AmpliTaq Gold DNA polymerase (PE Applied Biosystems, Foster City, Calif.). The reaction was incubated in a RoboCycler 40 Temperature Cycler (Stratagene Cloning Systems, La Jolla, Calif.) programmed for one cycle at 95° C. for 9 minutes; 30 cycles each at 95° C. for 1 minute, 55° C. for 1 minute, and 72° C. for 1 minute; and a final cycle at 72° C. for 7 minutes.

The reaction products were isolated on a 1% agarose gel (Eastman Kodak, Rochester, N.Y.) where a 320 bp product band was excised from the gel and purified using a Qiaquick DNA gel extraction kit (QIAGEN, Valencia, Calif.) according to the manufacturer's instructions. The 320 bp probe was designated RBggt-1/7R. As described in Example 5, this probe was shown to hybridized to the region of homology described in Example 1.

### Example 5

### Screening of Genomic Libraries

The RBggt1/7R probe (described in Example 4) was used to screen a size selected genomic library of Bacillus agaradhaerens AC13. The library was constructed by digesting Bacillus agaradhaerens AC13 chromosomal DNA (described in Example 3) with various restriction enzymes that do not cut within the region of homology described in Example 2.

Southern hybridization was performed using the DIG System for Filter Hybridization (Roche Diagnostics Corporation, Indianapolis, Ind.) according to the manufacturer's instruction. The blotting was performed using PosiBlot Pressure Blotter and Pressure Control Station (Stratagene Cloning Systems, La Jolla, Calif.) according to manufacturer's instructions. This Southern hybridization was performed using the digested Bacillus agaradhaerens AC 13 chromosomal DNA (and digested Bacillus licheniformis SJ3047 DNA as negative control) to determine the size of the fragments to be cloned in the library. The probe hybridized with a 2,500 bp HindIII fragment. Fragments of 2,000-3,000 bp were subsequently gel-purified (as described in Example 4) and Southern hybridization (as described above) was performed to confirm that the probe hybridized to the extracted fragments using the same conditions described above.

pUC18 was digested with HindIII and dephosphorylated using calf intestinal phosphatase (New England Biolabs Inc, Beverly, Mass.). The gel-purified 2,000-3,000 bp fragments were ligated to this plasmid using the Rapid DNA cloning kit (Roche Diagnostics Corporation, Indianapolis, Ind.) according to the manufacturer's instructions. Supercompetent Escherichia coli XL10-Gold cells (Stratagene Cloning Systems, La Jolla, Calif.) were transformed with the size selected genomic library. Ampicillin resistant transformants were selected on 2× YT plates (composed per liter of 16 g of Tryptone, 10 g of yeast extract, 5 g of sodium chloride, and 15 g of agar) supplemented with 100 μg of ampicillin per ml.

The transformants were screened by colony lifts using the same DIG-labeled probe RBggt1/7R following the Genius System instructions (Roche Diagnostics Corporation, Indianapolis, Ind.). After screening approximately 17,000 colonies, several potential positive clones were obtained. All the positive clones were identical since the library was constructed using a complete HindIII digest of the Bacillus agaradhaerens AC13 genomic DNA.

Plasmid DNA from a several of the positive clones was isolated using a Bio Robot 9600 (QIAGEN, Valencia, Calif.) according to the manufacturer's instructions. Restriction digests and PCR analyses (as described in Example 4) performed on the plasmid DNA indicated that the clones contained an insert of approximately 2,700 bp. Sequencing with an Applied Biosystems Model 3700 Automated DNA Sequencer (Perkin-Elmer/Applied Biosystems, Inc., Foster City, Calif.) using dye-terminator chemistry (Giesecke et al., 1992, Journal of Virology Methods 38: 47-60) and synthetic oligonucleotides based on the gamma-glutamyl transpeptidase gene sequence showed that only a 5′ partial clone was obtained. This clone included the ribosomal binding site and the first 914 bp of the Bacillus agaradhaerens gamma-glutamyl transpeptidase gene.

Since the size-selected HindIII library from Bacillus agaradhaerens AC13 genomic DNA provided only a 5′ partial clone of the gamma-glutamyl transpeptidase gene, a new cloning strategy was devised in order to obtain the full-length gamma-glutamyl transpeptidase gene. Since the Bacillus subtilis gamma-glutamyl transpeptidase homologue is 1,772 bp, and the 5′ partial clone encompassed the first 914 bp of the gamma-glutamyl transpeptidase gene, approximately 858 bp of the downstream gamma-glutamyl transpeptidase gene were missing. A fragment of at least 2,000 bp was necessary to ensure that the fragment encompassed the entire length of the gamma-glutamyl transpeptidase gene. Southern hybridizations (using the RBggt1/7R probe as described above) were performed using Bacillus agaradhaerens AC13 genomic DNA digested with several restriction enzymes and combinations thereof.

A Southern blot of PvuII/SacI digested genomic DNA provided a band that was about 2,500 bp (PvuII cuts 124 bp upstream of the gamma-glutamyl transpeptidase gene's RBS). Consequently, 2,000-3,000 bp fragments of the PvuII/SacI digested genomic DNA were gel-purified (as described in Example 4). Southern hybridization (as described above) confirmed that the ggt probe hybridized to the extracted fragments. Approximately 2,000-3,000 bp PvuII/SacI fragments were ligated (as described above) to pUC19 digested with HindIII/SacI. Supercompetent Escherichia coli XL10-Gold cells were transformed with the ligation mixture and ampicillin resistant transformants were selected on 2× YT plates supplemented with 100 μg of ampicillin per ml.

Colony lifts and hybridizations were performed using the same DIG-labeled probe RBggt1/7R following the Genius System instructions (as described above). Numerous potential positive clones were obtained. Again, all these positive clones were likely identical since the library was constructed using a complete PvuII/SacI double digest of Bacillus agaradhaerens genomic DNA.

Plasmid DNA from several of the positive clones was isolated using a Bio Robot 9600. Restriction, PCR (as described in Example 4), and sequencing analyses (as described in Example 5) were performed on one of these clones, which showed that this clone was partial containing the 3′ end of the gamma-glutamyl transpeptidase gene. This clone was missing the first 250 bp from the start codon, yet contained the stop codon and terminator of the Bacillus agaradhaerens gamma-glutamyl transpeptidase gene.

The 3′ and 5′ partial clones were then pieced together in order to obtain the full-length gamma-glutamyl transpeptidase gene as described in Example 6.

### Example 6

### Reconstruction of the Bacillus agaradhaerens Gamma-Glutamyl Transpeptidase Gene

The pUC18/5′ gamma-glutamyl transpeptidase gene clone was digested with PvuII and BglII in order to isolate the 5′ end of the gene. The pUC19/3′ gamma-glutamyl transpeptidase gene clone was digested with BglII and EcoRI and filled-in with T4 DNA polymerase (Roche Diagnostics Corporation, Indianapolis, Ind.) in order to isolate the vector and the 3′ end of the gene. The 5′ and 3′ fragments were ligated together using the Rapid DNA cloning kit (Roche Diagnostics Corporation, Indianapolis, Ind.) according to the manufacturer's instructions. Competent Escherichia coli XLI-Blue cells (Stratagene Cloning Systems, La Jolla, Calif.) were transformed with 2 μl of the ligation mixture according to the manufacturer's instructions. Ampicillin resistant transformants were selected on 2× YT plates supplemented with 100 μg of ampicillin per ml. Plasmid DNA from several of the positive clones was isolated using a Bio Robot 9600. Restriction analyses were performed on several of the transformants to confirm the presence of the full-length gamma-glutamyl transpeptidase gene. One positive E. coli XLI-Blue (pUC19/ggt) transformant was designated E. coli RB155.

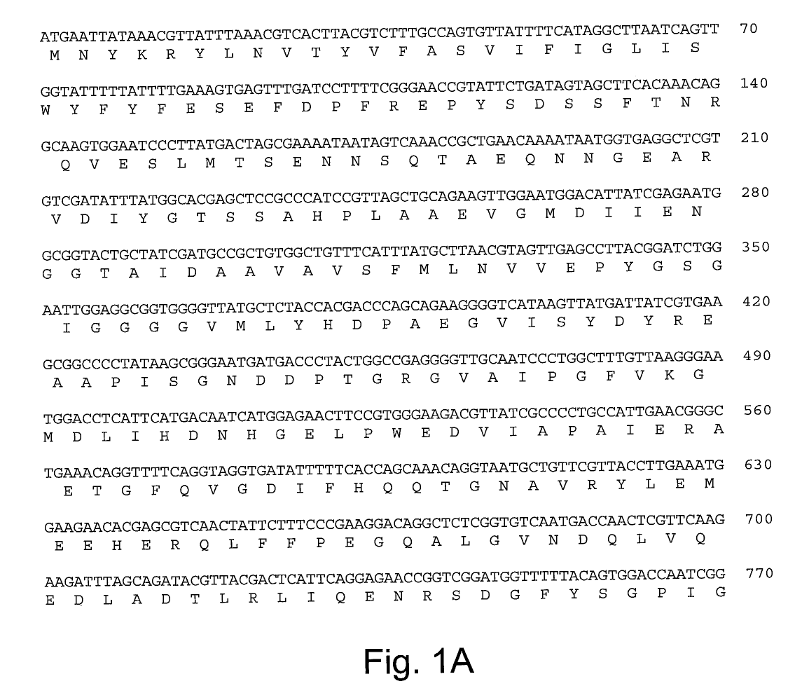

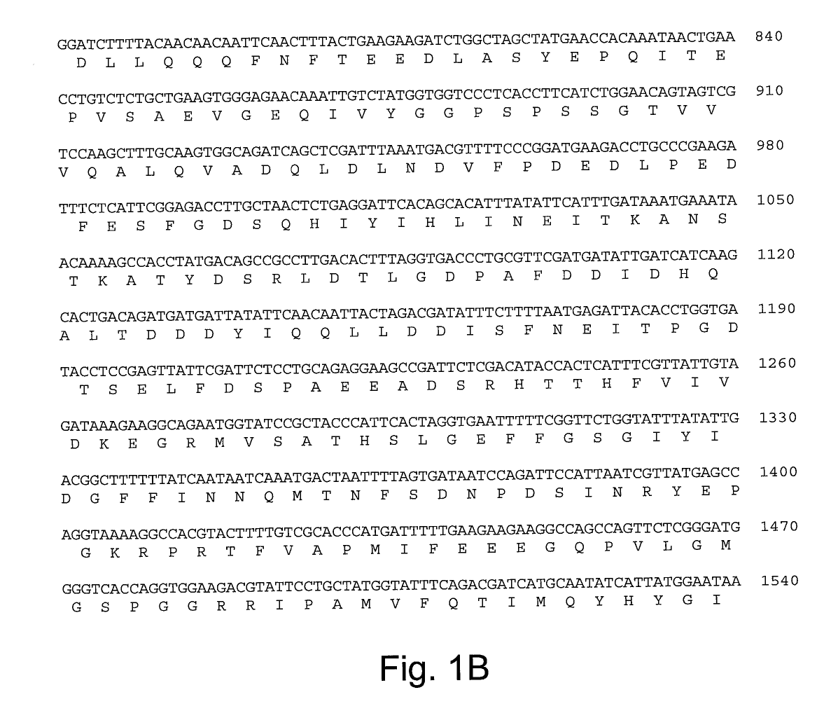

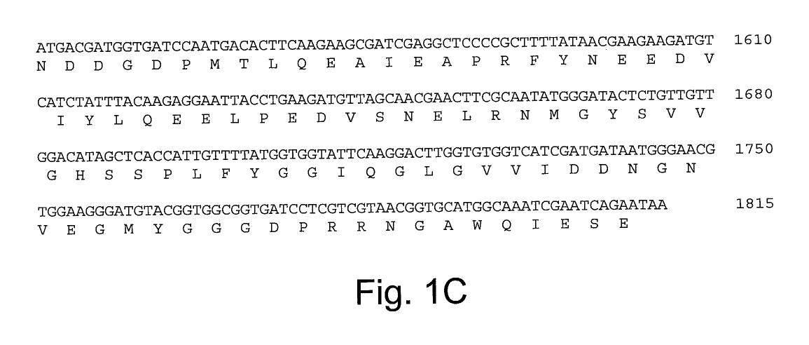

The gamma-glutamyl transpeptidase gene was sequenced with an Applied Biosystems Model 3700 Automated DNA Sequencer using dye-terminator chemistry and synthetic oligonucleotides based on the gamma-glutamyl transpeptidase gene sequence. The nucleic acid sequence (SEQ ID NO. 1) and the deduced amino acid sequence (SEQ ID NO. 2) are shown in FIG. 1.

E. coli RB155 was deposited with the NRRL Culture collection (NRRL# B-30340).

### Example 7

### Isolation and Characterization of Gamma-Glutamyl Transpeptidase Gene from Bacillus agaradhaerens AC13

Oligonucleotide primers RBggtBamHI and RBggtNotI shown below were used to amplify the gamma-glutamyl transpeptidase coding region from pUC19/ggt (described in Example 6) by PCR. Primer RBggtBamHI incorporated a BamHI site (see underlined nucleotides below) and the ribosome-binding site of the gamma-glutamyl transpeptidase gene upstream of the gamma-glutamyl transpeptidase coding region, and primer RBggtNotI incorporated a NotI site (see underlined nucleotides below) downstream of the gamma-glutamyl transpeptidase coding region.

Primer RBggtBamHI:

| | |
|---|---|
| 5′-TTGGATCCGTACTTGAAGGAGTGGACTTAACCTCATGAATTATAAA-3′ | (SEQ ID NO.5) |

Primer RBggtNotI:

| | |
|---|---|
| 5′-GGCGGCCGCGATGTGTTTACAGGATAACGCTCGTAAAGTTAA-3′ | (SEQ ID NO.6) |

The amplification reaction (50 μl) contained approximately 50 ng of pUC19/ggt plasmid DNA, 0.5 μM of each primer, 200 μM each of dATP, dCTP, dGTP, and dTTP, 1× PCR buffer, 3 mM MgCl₂, and 0.625 units of AmpliTaq Gold DNA polymerase (PE Applied Biosystems, Foster City, Calif.). The reaction was incubated in a RoboCycler 40 Temperature Cycler programmed for one cycle at 95° C. for 9 minutes; 30 cycles each at 95° C. for 1 minute, 55° C. for 1 minute, and 72° C. for 2 minutes; and a final cycle at 72° C. for 7 minutes.

The PCR product of approximately 1,941 bp was gel-purified using a Qiaquick gel extraction kit.

The PCR product was then cloned using a TOPO TA Cloning Kit (Invitrogen, Carlsbad, Calif.) according to the manufacturer's instructions. Ampicillin resistant transformants were selected on 2× YT plates supplemented with 100 μg of ampicillin per ml. Plasmid DNA was isolated from the Escherichia coli TOP10 transformants using a Bio Robot 9600 according to manufacturer's instructions. A plasmid containing the desired insert was identified by restriction analysis using EcoRI and was designated pCR2.1-ggt. The Escherichia coli TOP10 colony containing the pCR2.1-ggt plasmid was isolated, and plasmid DNA was prepared using a QIAGEN Plasmid Midi Kit (QIAGEN, Valencia, Calif.) according to the manufacturer's instructions.

DNA sequencing was performed with an Applied Biosystems Model 3700 Automated DNA Sequencer using dye-terminator chemistry and synthetic oligonucleotides based on the gamma-glutamyl transpeptidase gene sequence (as described in Example 5). DNA sequence analysis confirmed the sequence of the gene according to previous sequencing results (described in Examples 5 and 6).

The gamma-glutamyl transpeptidase gene has an open reading frame of 1,812 bp encoding a polypeptide of 604 amino acids. The nucleotide sequence (SEQ ID NO. 1) and deduced amino acid sequence (SEQ ID NO. 2) are shown in FIG. 1. Using the SignalP program (Nielsen et al., 1997, Protein Engineering 10: 1-6), a signal peptide of 30 residues was predicted corresponding to nucleotides 1 to 90 of SEQ ID NO. 1.

A comparative alignment of the gamma-glutamyl transpeptidase amino acid sequence was determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters were Ktuple=1, gap penalty=3, windows=5, and diagonals=5.

The comparative alignment showed that the Bacillus agaradhaerens gamma-glutamyl transpeptidase shared regions of identity of 24.0% with the gamma-glutamyl transpeptidase from Escherichia coli (EMBL M28722) and 22.7% with the gamma-glutamyl transpeptidase from Bacillus subtilis (EMBL U49358).

### Example 8

### Construction of pDG268MCSdeltaneo/scBAN/cry3Alongstabilizer/ggt

pDG268MCSΔneo/scBAN/cry3A long stabilizer/SAVINASE™ was constructed as described in U.S. Pat. No. 5,955,310. The plasmid was phenol extracted twice and ethanol precipitated prior to being digested with Ecl136II and NotI. The largest plasmid fragment of approximately 7,400 bp was gel-purified using a Qiaquick DNA gel extraction kit according to the manufacturer's instructions. The recovered vector DNA was then ligated with the insert DNA described below.

pCR2.1TOPO/ggt (described in Example 7) was digested with BamHI, treated with T4 DNA polymerase (Roche Diagnostics Corporation, Indianapolis, Ind.) and dNTPs according to the manufacturer's instructions to generate blunt ends. The digested plasmid DNA was then purified using a Qiaquick DNA purification kit (QIAGEN, Valencia, Calif.) according to the manufacturer's instructions, and finally digested with NotI. The smallest plasmid fragment of approximately 1,934 bp was gel-purified using a Qiaquick DNA gel extraction kit according to the manufacturer's instructions.

The recovered vector and insert DNA were ligated using the Rapid DNA cloning kit (Roche Diagnostics Corporation, Indianapolis, Ind.) according to the manufacturer's instructions. Competent Escherichia coli SURE cells (Stratagene Cloning Systems, La Jolla, Calif.) were transformed with this ligation mixture according to the manufacturer's instructions. Escherichia coli ampicillin resistant transformants were selected on 2× YT plates supplemented with 100 μg of ampicillin per ml. Plasmid DNA was isolated from the ampicillin resistant transformants using a Bio Robot 9600 according to manufacturer's instructions. A plasmid containing the desired gamma-glutamyl transpeptidase gene insert was identified by restriction analysis using PstI and was designated pDG268MCSΔneo/scBAN/cry3A long stabilizer/ggt. Plasmid DNA from this positive clone was prepared using a QIAGEN Plasmid Midi Kit according to the manufacturer's instructions.

### Example 9

### Cloning of Gamma-Glutamyl Transpeptidase Gene in Bacillus subtilis A164delta5

Competent Bacillus subtilis 164Δ5 (WO 98/22598) were transformed with pDG268MCSΔneo/scBAN/cry3A long stabilizer/ggt linearized with the restriction enzyme ScaI. Bacillus subtilis chloramphenicol resistant transformants were selected on Tryptose Blood Agar Base (TBAB) plates supplemented with 5 μg of chloramphenicol per ml. To screen for integration of the plasmid by double cross-over at the amyE locus, Bacillus subtilis transformants were patched on TBAB plates supplemented with 6 μg of neomycin per ml and on TBAB plates supplemented with 5 μg of chloramphenicol per ml. Integration of the plasmid by double cross-over at the amyE locus loses the neomycin resistance gene and renders the strain neomycin sensitive. One such Bacillus subtilis A164Δ5 ggt integrant was isolated and designated Bacillus subtilis RB157.

Genomic DNA was isolated from Bacillus subtilis RB157 using the QIAGEN bacterial genomic DNA isolation protocol (QIAGEN, Valencia, Calif.) according to the manufacturer's instructions. A plate assay to check the phenotype of this new gamma-glutamyl transpeptidase strain was not available. Consequently, RB157 genomic DNA was used to perform PCR amplifications to confirm the presence and integrity of the gamma-glutamyl transpeptidase gene.

The amplification reaction (25 μl) contained approximately 50 ng of genomic DNA, 0.5 μM of each primer, 200 μM each of dATP, dCTP, dGTP, and dTTP, 1× PCR buffer, 3 mM MgCl₂, and 0.625 units of AmpliTaq Gold DNA polymerase (PE Applied Biosystems, Foster City, Calif.). The reactions were incubated in a RoboCycler 40 Temperature Cycler programmed as described in Example 4.

Synthetic oligonucleotides based on the gamma-glutamyl transpeptidase gene sequence confirmed the presence and integrity of the gamma-glutamyl transpeptidase gene in this final Bacillus subtilis strain RB157.

### Deposit of Biological Material

The following biological material has been deposited under the terms of the Budapest Treaty with the Agricultural Research Service Patent Culture Collection, Northern Regional Research Center, 1815 University Street, Peoria, Ill., 61604, and given the following accession number:

| Deposit | Accession Number | Date of Deposit |
|---|---|---|
| E. coli RB 155 | NRRL B-30340 | Oct. 4, 2000 |

The strain has been deposited under conditions that assure that access to the culture will be available during the pendency of this patent application to one determined by the Commissioner of Patents and Trademarks to be entitled thereto under 37 C.F.R. §1.14 and 35 U.S.C. §122. The deposit represents a substantially pure culture of the deposited strain. The deposit is available as required by foreign patent laws in countries wherein counterparts of the subject application, or its progeny are filed. However, it should be understood that the availability of a deposit does not constitute a license to practice the subject invention in derogation of patent rights granted by governmental action.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

What is claimed is:

1. An isolated polypeptide having gamma-glutamyl transpeptidase activity, selected from the group consisting of:
(a) a polypeptide having an amino acid sequence which has at least 65% identity with amino acids 31 to 604 for the mature polypeptide of SEQ ID NO. 2;
(b) a polypeptide which is encoded by a nucleic acid sequence which hybridizes under low stringency conditions with (i) (i) nucleotides 91 to 1812 of SEQ ID NO. 1, (ii) a subsequence of (i) or (ii) of at least 100 nucleotides, or (iii) a complementary strand of (i) or (ii); and
(c) a fragment of (a) or (b), that has gamma-glutamyl transpeptidase activity.

2. The polypeptide of claim 1, having an amino acid sequence which has at least 65% identity with amino acids 31 to 604 of SEQ ID NO. 2.

3. The polypeptide of claim 2, having an amino acid sequence which has at least 70% identity with amino acids 31 to 604 of SEQ ID NO. 2.

4. The polypeptide of claim 3, having an amino acid sequence which has at least 80% identity with amino acids 31 to 604 of SEQ ID NO. 2.

5. The polypeptide of claim 4, having an amino acid sequence which has at least 90% identity with amino acids 31 to 604 of SEQ ID NO. 2.

6. The polypeptide of claim 5, having an amino acid sequence which has at least 95% identity with amino acids 31 to 604 of SEQ ID NO. 2.

7. The polypeptide of claim 1, comprising the amino acid sequence of SEQ ID NO. 2.

8. The polypeptide of claim 1, consisting of the amino acid sequence of SEQ ID NO. 2 or a fragment thereof.

9. The polypeptide of claim 8, consisting of the amino acid sequence of SEQ ID NO. 2.

10. The polypeptide of claim 9, which consists of amino acids 31 to 604 of SEQ ID NO. 2.

11. The polypeptide of claim 1, which is encoded by a nucleic acid sequence which hybridizes under low stringency conditions with (i) nucleotides 91 to 1812 of SEQ ID NO. 1, (ii) a subsequence of (i) or (ii) of at least 100 nucleotides, or (iii) a complementary strand of (i) or (ii).

12. The polypeptide of claim 11, which is encoded by a nucleic acid sequence which hybridizes under low stringency conditions with nucleotides 91 to 1812 of SEQ ID NO. 1, or its complementary strand.

13. The polypeptide of claim 1, which is encoded by a nucleic acid sequence which hybridizes under medium stringency conditions with (i) nucleotides 91 to 1812 of SEQ ID NO. 1, (ii) a subsequence of (i) or (ii) of at least 100 nucleotides, or (iii) a complementary strand of (i) or (ii).

14. The polypeptide of claim 13, which is encoded by a nucleic acid sequence which hybridizes under medium stringency conditions with nucleotides 91 to 1812 of SEQ ID NO. 1, or its complementary strand.

15. The polypeptide of claim 1, which is encoded by a nucleic acid sequence which hybridizes under high stringency conditions with (i) nucleotides 91 to 1812 of SEQ ID NO. 1, (ii) a subsequence of (i) or (ii) of at least 100 nucleotides, or (iii) a complementary strand of (i) or (ii).

16. The polypeptide of claim 15, which is encoded by a nucleic acid sequence which hybridizes under high stringency conditions with nucleotides 91 to 1812 of SEQ ID NO. 1, or its complementary strand.

17. The polypeptide of claim 1, which is encoded by the nucleic acid sequence contained in plasmid pRB155 which is contained in E. coli NRRL B-30340.

18. The polypeptide of claim 1, which has at least 20% of the gamma-glutamyl transpeptidase activity of SEQ ID NO. 2.

19. A polypeptide having the same gamma-glutamyl transpeptidase activity as the polypeptide of claim 1.

20. An isolated nucleic acid sequence comprising a nucleic acid sequence which encodes the polypeptide of claim 1.

21. An isolated nucleic acid sequence comprising a nucleic acid sequence having at least one mutation in the mature polypeptide coding sequence of SEQ ID NO. 1, in which the mutant nucleic acid sequence encodes a polypeptide consisting of amino acids 31 to 604 of SEQ ID NO. 2.

22. An isolated nucleic acid sequence produced by (a) hybridizing a DNA under low stringency conditions with (i) nucleotides 91 to 1812 of SEQ ID NO. 1, (ii) a subsequence of (i) or (ii) of at least 100 nucleotides, or (iii) a complementary strand of (i) or (ii); and (b) isolating the nucleic acid sequence.

23. The isolated nucleic acid sequence of claim 22 produced by (a) hybridizing a DNA under medium stringency conditions with (i) nucleotides 91 to 1812 of SEQ ID NO. 1, (ii) a subsequence of (i) or (ii) of at least 100 nucleotides, or (iii) a complementary strand of (i) or (ii); and (b) isolating the nucleic acid sequence.

24. An isolated nucleic acid sequence of claim 23 produced by (a) hybridizing a DNA under high stringency conditions with (i) nucleotides 91 to 1812 of SEQ ID NO. 1, (ii) a subsequence of (i) or (ii) of at least 100 nucleotides, or (iii) a complementary strand of (i) or (ii); and (b) isolating the nucleic acid sequence.

25. A nucleic acid construct comprising the nucleic acid sequence of claim 20 operably linked to one or more control sequences that direct the production of the polypeptide in a suitable expression host.

26. A recombinant expression vector comprising the nucleic acid construct of claim 25.

27. A recombinant host cell comprising the nucleic acid construct of claim 25.

28. A method for producing a mutant nucleic acid sequence, comprising (a) introducing at least one mutation into the mature polypeptide coding sequence of SEQ ID NO. 1, wherein the mutant nucleic acid sequence encodes a polypeptide consisting of amino acids 31 to 604 of SEQ ID NO. 2; and (b) recovering the mutant nucleic acid sequence.

29. A mutant nucleic acid sequence produced by the method of claim 28.

30. A method for producing a polypeptide, comprising (a) cultivating a strain comprising the mutant nucleic acid sequence of claim 29 encoding the polypeptide to produce a supernatant comprising the polypeptide; and (b) recovering the polypeptide.

31. A method for producing the polypeptide of claim 1 comprising (a) cultivating a strain to produce a supernatant comprising the polypeptide; and (b) recovering the polypeptide.

32. A method for producing the polypeptide of claim 1 comprising (a) cultivating a host cell comprising a nucleic acid construct comprising a nucleic acid sequence encoding the polypeptide under conditions suitable for production of the polypeptide; and (b) recovering the polypeptide.

33. A method for producing a polypeptide comprising (a) cultivating a host cell under conditions conducive for production of the polypeptide, wherein the host cell comprises a mutant nucleic acid sequence having at least one mutation in the mature polypeptide coding sequence of SEQ ID NO. 1, wherein the mutant nucleic acid sequence encodes a polypeptide consisting of amino acids 31 to 604 of SEQ ID NO. 2, and (b) recovering the polypeptide.

34. A method for producing a mutant of a cell, which comprises disrupting or deleting a nucleic acid sequence encoding the polypeptide of claim 1 or a control sequence thereof, which results in the mutant producing less of the polypeptide than the cell.

35. A mutant produced by the method of claim 34.

36. The mutant of claim 35, which further comprises a nucleic acid sequence encoding a heterologous protein.

37. A method for producing a heterologous polypeptide comprising (a) cultivating the mutant of claim 36 under conditions conducive for production of the polypeptide; and (b) recovering the polypeptide.

38. A nucleic acid construct comprising a gene encoding a protein operably linked to a nucleic acid sequence encoding a signal peptide consisting of nucleotides 1 to 90 of SEQ ID NO. 1, wherein the gene is foreign to the nucleic acid sequence.

39. A recombinant expression vector comprising the nucleic acid construct of claim 38.

40. A recombinant host cell comprising the nucleic acid construct of claim 38.

41. A method for producing a protein comprising (a) cultivating the recombinant host cell of claim 40 under conditions suitable for production of the protein; and (b) recovering the protein.


SEQUENCE LISTING (6 sequences)

SEQ ID NO: 1 - DNA, 1815 bp, Bacillus agaradhaerens
atgaattataaacgttatttaaacgtcacttacgtctttgccagtgttattttcataggcttaatcagttggtatttttattttgaaagtgagtttgatccttttcgggaaccgtattctgatagtagcttcacaaacaggcaagtggaatcccttatgactagcgaaaataatagtcaaaccgctgaacaaaataatggtgaggctcgtgtcgatatttatggcacgagctccgcccatccgttagctgcagaagttggaatggacattatcgagaatggcggtactgctatcgatgccgctgtggctgtttcatttatgcttaacgtagttgagccttacggatctggaattggaggcggtggggttatgctctaccacgacccagcagaaggggtcataagttatgattatcgtgaagcggcccctataagcgggaatgatgaccctactggccgaggggttgcaatccctggctttgttaagggaatggacctcattcatgacaatcatggagaacttccgtgggaagacgttatcgcccctgccattgaacgggctgaaacaggttttcaggtaggtgatatttttcaccagcaaacaggtaatgctgttcgttaccttgaaatggaagaacacgagcgtcaactattctttcccgaaggacaggctctcggtgtcaatgaccaactcgttcaagaagatttagcagatacgttacgactcattcaggagaaccggtcggatggtttttacagtggaccaatcggggatcttttacaacaacaattcaactttactgaagaagatctggctagctatgaaccacaaataactgaacctgtctctgctgaagtgggagaacaaattgtctatggtggtccctcaccttcatctggaacagtagtcgtccaagctttgcaagtggcagatcagctcgatttaaatgacgttttcccggatgaagacctgcccgaagatttctcattcggagaccttgctaactctgaggattcacagcacatttatattcatttgataaatgaaataacaaaagccacctatgacagccgccttgacactttaggtgaccctgcgttcgatgatattgatcatcaagcactgacagatgatgattatattcaacaattactagacgatatttcttttaatgagattacacctggtgatacctccgagttattcgattctcctgcagaggaagccgattctcgacataccactcatttcgttattgtagataaagaaggcagaatggtatccgctacccattcactaggtgaatttttcggttctggtatttatattgacggcttttttatcaataatcaaatgactaattttagtgataatccagattccattaatcgttatgagccaggtaaaaggccacgtacttttgtcgcacccatgatttttgaagaagaaggccagccagttctcgggatggggtcaccaggtggaagacgtattcctgctatggtatttcagacgatcatgcaatatcattatggaataaatgacgatggtgatccaatgacacttcaagaagcgatcgaggctccccgcttttataacgaagaagatgtcatctatttacaagaggaattacctgaagatgttagcaacgaacttcgcaatatgggatactctgttgttggacatagctcaccattgttttatggtggtattcaaggacttggtgtggtcatcgatgataatgggaacgtggaagggatgtacggtggcggtgatcctcgtcgtaacggtgcatggcaaatcgaatcagaataa

SEQ ID NO: 2 - protein, 604 aa, Bacillus agaradhaerens
MNYKRYLNVTYVFASVIFIGLISWYFYFESEFDPFREPYSDSSFTNRQVESLMTSENNSQTAEQNNGEARVDIYGTSSAHPLAAEVGMDIIENGGTAIDAAVAVSFMLNVVEPYGSGIGGGGVMLYHDPAEGVISYDYREAAPISGNDDPTGRGVAIPGFVKGMDLIHDNHGELPWEDVIAPAIERAETGFQVGDIFHQQTGNAVRYLEMEEHERQLFFPEGQALGVNDQLVQEDLADTLRLIQENRSDGFYSGPIGDLLQQQFNFTEEDLASYEPQITEPVSAEVGEQIVYGGPSPSSGTVVVQALQVADQLDLNDVFPDEDLPEDFESFGDSQHIYIHLINEITKANSTKATYDSRLDTLGDPAFDDIDHQALTDDDYIQQLLDDISFNEITPGDTSELFDSPAEEADSRHTTHFVIVDKEGRMVSATHSLGEFFGSGIYIDGFFINNQMTNFSDNPDSINRYEPGKRPRTFVAPMIFEEEGQPVLGMGSPGGRRIPAMVFQTIMQYHYGINDDGDPMTLQEAIEAPRFYNEEDVIYLQEELPEDVSNELRNMGYSVVGHSSPLFYGGIQGLGVVIDDNGNVEGMYGGGDPRRNGAWQIESE

SEQ ID NO: 3 - DNA, 20 bp, Bacillus agaradhaerens
gcccatccgttagctgcaga

SEQ ID NO: 4 - DNA, 21 bp, Bacillus agaradhaerens
ttcaatggcaggggcgataac

SEQ ID NO: 5 - DNA, 45 bp, Bacillus agaradhaerens
tggatccgtacttgaaggagtggacttaacctcatgaattataaa

SEQ ID NO: 6 - DNA, 42 bp, Bacillus agaradhaerens
ggcggccgcgatgtgtttacaggataacgctcgtaaagttaa